(19) United States
(12) Patent Application Publication
(10) Pub. No.: US 2017/0127973 A1
(43) Pub. Date: May 11, 2017

(54) DEVICE, SYSTEM AND METHOD FOR MEASURING ESOPHAGEAL MUCOSAL IMPEDANCE

(71) Applicant: Covidien LP, Mansfield, MA (US)

(72) Inventors: Thomas R. PARKS, Mammoth Lakes, CA (US); Benjamin James Heath PAGLIUSO, Culver City, CA (US)

(73) Assignee: Covidien LP, Mansfield, MA (US)

(21) Appl. No.: 15/345,909

(22) Filed: Nov. 8, 2016

Related U.S. Application Data
(60) Provisional application No. 62/253,728, filed on Nov. 11, 2015.

(51) Int. Cl. A61B 5/053; A61B 5/00

(52) U.S. Cl. CPC A61B 5/0538; A61B 5/6859; A61B 5/4211; A61B 5/687

(57) ABSTRACT

An mucosal impedance (“MI”) device may include or utilize a retaining member sized to retain a tines structure including N tines, each provided with one or more impedance electrodes on each tine. The retaining member may mechanically deform the tines such that they fit inside it. The tines, made of spring like material, may be chosen, for example, for their elastic modulus and yield strength. The tines may be pre-conditioned with a shape that provides a spring tension force in the radial outward direction. When the retaining member or is retracted to release and expose the tines and impedance electrodes, the preconditioned shape and elasticity of the tines cause the tines to expand radially outward, providing an appropriate contact force of the impedance electrodes on the inner wall of the esophagus for a range of esophageal diameters.

### PRIOR APPLICATION DATA

This application claims benefit from prior provisional patent application Ser. No. 62/253,728, filed Nov. 11, 2015, entitled “DEVICE, SYSTEM AND METHOD FOR MEASURING ESOPHAGEAL MUCOSAL IMPEDANCE”, incorporated by reference herein in its entirety.

### FIELD OF THE INVENTION

The present invention generally relates to catheters and more specifically to a device, system and method for measuring esophageal mucosal impedance for assessing damages caused to the esophageal mucosa, for example, by gastric reflux.

### BACKGROUND

Gastroesophageal reflux disease (“GERD”) is a disease caused by gastric reflux. Since a main cause for GERD is a malfunctioning LES (lower esophageal sphincter) that enables acid content to flow backwards from the stomach to the esophagus, assessing the severity of GERD has traditionally involved direct pH measurements in the esophagus. However, using pH measurements to assess GERD, or other pH-induced damages is problematic in at least two aspects, the first aspect being that while measuring pH can indicate the presence of acidic fluids in the esophagus, it does not provide a direct indication as to the actual damage that such fluids cause to the esophagus tissue. (That is, tissue damage can only be inferred from pH measurements.) The second aspect is that pH measurements typically need to be acquired in the esophagus over a period of time in order to try assess a damage caused to the esophageal tissue by gastric fluids. That is, while occasional gastric reflux may have little impact on the esophageal tissue, frequent gastric reflux may cause severe damage to the esophageal tissue. Recent endeavors have been directed to measuring electrical impedance (Z) of the esophageal tissue, because electrical impedance of a tissue is a direct, and therefore more reliable, indication to a damage inflicted to esophageal tissue by gastric fluids and by other factors.

FIG. 1A and FIG. 1B show two example esophageal tissue impedance-measurements setups. Referring to FIG. 1A, into esophagus 100 is inserted a pH catheter 110 on which five impedance rings 120 are mounted for measuring impedance of various tissue areas of/in esophagus 100. (Upper Esophageal Sphincter (“UES”), Lower Esophageal Sphincter (“LES”) and the stomach are respectively shown at 140, 150 and 160.) Referring to FIG. 1B, into esophagus 100 is inserted an endoscope 102 from which mucosal impedance catheter 104 is pushed, through a working channel of/in endoscope 102 until two impedance electrodes 106 (one electrode pair 106) touch(s) the esophageal mucosa whose impedance is (or to be) measured.

FIG. 1C (prior art) proposes another impedance measuring system. A balloon 180 includes multiple electrodes 190 on its periphery. In order to measure esophageal impedances, balloon 180 has to be inflated, as shown in FIG. 1C. Balloon 180 is central and electrodes 190 are distributed at multiple radial positions on the external surface of balloon 180, which, in operation, pushes the electrodes into contact with the wall (182) of the esophagus.

Using the impedance measurement setups of FIGS. 1A-1C may be restrictive because using these setups requires an inflatable object (170; 180) to press impedance rings 120 (per FIG. 1A), or impedance electrodes 106 (per FIG. 1B) or impedance electrodes 190 (per FIG. 1C) against the esophageal tissue in order to make good contact therewith. Using inflatable objects 170 and 180 may be problematic, for example, because using them involves complexity of operation and requires air/gas supply equipment (to inflate the inflatable objects). In addition, the inflatable objects have a relatively large cross sectional area for introduction of the device. This relatively large cross sectional area may be restrictive when attempting to deploy the device through a working channel of, for example, an endoscope. In addition, since inflatable object 170 pushes the impedance rings/electrodes in one direction, the tissue's impedance can be measured only at one side of the esophageal tissue at a time, which means that the whole measurement setup has to be maneuvered (e.g., axially rotated) after each measurement in order to measure other sides (other peripheral points/areas) of the esophageal tissue. When inflatable object 180 is fully inflated, it completely blocks the esophagus. This may cause discomfort to the patient and prevent insertion of additional devices into the esophagus. For example, it may be required or desired to visually assure (e.g., by inserting a camera device into the esophagus) that the electrodes are deployed at the accurate location in the esophagus. However, using a balloon may obscure the view.

### SUMMARY

While measuring mucosal impedance (“MI”) of esophageal tissue is beneficial in estimating the severity of a damage caused to the esophageal tissue, it would be beneficial to have a MI device that simultaneously measures lengthwise impedances, lateral impedances and diagonal impedances of the tissue at multiple peripheral points/areas, and, in addition, would do that without requiring an extrinsic device (e.g., an inflatable object) to assure reliable contact between the impedance electrodes and the esophageal mucosa.

An MI device of the present invention may include a retaining member (e.g., a sheath) or utilize one (e.g., an external tubular instrument) which is sized to contain N tines/ribs structures. The N tines may be provided with one or more impedance electrodes on each tine, and the MI device, as a whole, may be sized to fit through a working channel, for example, of an endoscope. The retaining member may mechanically deform (e.g., coil) the tines such that they would fit into/inside the retaining member. The tines, made of suitable elastic material, may be chosen for, as an example, their elastic modulus and yield strength. The tines may be pre-conditioned with a shape and mechanical properties that provides a spring tension force in a radial outward direction (with respect to a longitudinal axis of the retaining member or MI device). When the retaining member is retracted to expose the tines and the impedance electrodes that are provided on them, the preconditioned shape and elasticity of the tines may cause the tines structure to expand radially outward (with respect to the longitudinal axis of the retaining member or MI device as a whole) to provide an appropriate minimum contact force for pushing impedance electrodes against the inner wall of the esophageal mucosa for a range of esophageal sizes or diameters. The retaining member may be a working channel of an endoscope.

Electrodes mounted in or on the tines may be selected to measure lengthwise impedances, lateral impedances and diagonal impedances of esophageal tissue. (A ‘lengthwise impedance’ is an impedance measured between two electrodes that are located on the same tine. A ‘lateral impedance’ is an impedance measured between two electrodes that are located on different tines and a line passing through them is perpendicular, or substantially perpendicular, to the tines. A ‘diagonal impedance’ is an impedance measured between two electrodes that are located on different tines and a line passing through them is at angle substantially different than 90 degrees with respect to the tines.)

In some embodiments the MI device may be used without an endoscope. For example, it may be placed trans-nasally by, for example, a nurse in a similar manner as is done with, for example, manometry and pH tests in the esophagus (as an example). Placement of an MI device may also be done trans-orally without an endoscope.

In some embodiments the N tines or ribs of the MI device may form an ‘open-sided’ flexible structure. The term ‘open-sided structure’, as used herein, refers to a tines structure having a closed end where the N flexible tines are firmly tight together (converge) at one end, and an open end formed by the other ends of the N flexible tines, which are capable of opening up due to the flexible tines being pre-conditioned or pre-shaped to deflect laterally outwardly when no external force is exerted on the tines (that is, in their tension-free or free state).

In other embodiments the N tines of the MI device may form a ‘closed-sided’ flexible tines structure. (The term ‘closed-sided structure’, as used herein, refers to a tines structure having a first closed end where the N flexible tines are firmly held together (at the ‘trailing’ or proximal end of the closed-sided structure) and also a second closed end where the N flexible tines are firmly held together (at the ‘leading’ or distal end of the closed-sided structure).

The MI device or retaining member may include an axial actuating member (“AAM”), which may be or may include, for example, a cord, a string, a wire, a shaft, and the like. (By “AAM” is meant a tension or compression member that is capable of or configured to transmit force, and/or cause lengthwise displacement, through or by it, without interfering with the flexibility of the device). The AAM may pass in or through the trailing end of the closed-ended tines structure and be connected to the leading or distal end of the tines structure. The AAM may move the tines structure, or MI device as a whole, relative to the retaining member, to a desired location. The MI device may include a force adjusting member (“FAM”) to adjust the force that the impedance electrodes apply to the mucosal wall by adjusting an operational diameter of the tines structure. Pulling or pushing the FAM may change the dimensions of (e.g., ‘open-up’) the closed-sided tines structure (e.g., overall length and/or diameter), and thus may enable controlling the contact pressure between the impedance electrodes on each tine and the esophageal mucosa. In some embodiments, a diameter of the tines structure in the free state is adjustable, for example, at least to some degree, to change a contact force of the open-sided or closed-sided variant. The AAM may control the lengthwise position of the tines structure, or one of its ends, which, in turn, may control the contact pressure of the impedance electrodes once the tines are in contact with the esophageal tissue. The AAM may be instrumental only in deploying and stowing (retracting) the tines, or it may additionally be instrumental in adjusting the force that the tines apply to the mucosal wall, though the latter (‘force’) function may be executed by the FAM.

A tine may have mechanical characteristics (e.g., spring constant) that change along its length in order to impart different flexibilities to different segments of the tine, or to impart different forces to different impedance electrodes against the mucosa based on their position on the tine or, in the same manner, create a more uniform force distribution to each electrode along the tine. The invention also includes a method for using the MI device and a system that implements the method.

### BRIEF DESCRIPTION OF THE DRAWINGS

Various exemplary embodiments are illustrated in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate corresponding, like, or analogous elements. Of the accompanying figures:

FIGS. 1A-1C (prior art) show typical mucosal impedance devices for measuring electrical impedance of an esophageal tissue;

FIGS. 2A-2C show a mucosal impedance (MI) device in various states according to an example embodiment of the invention;

FIG. 3 shows the MI device of FIGS. 2A-2C deployed in the esophagus according to an example embodiment of the invention;

FIG. 4 shows an example flexible tine or rib of an MI device according to an example embodiment of the invention;

FIG. 5 shows a mucosal impedance (MI) device according to another example embodiment of the invention;

FIGS. 6A-6B depict a mucosal impedance (MI) device similar to the device of FIG. 5 in two states thereof relative to an example esophagus;

FIG. 7 shows a mucosal impedance (MI) device with an adjustment cord according to another example embodiment of the invention;

FIG. 8 shows a control system for measuring mucosal impedances according to an example embodiment of the invention; and

FIG. 9 shows a method for measuring mucosal impedance according to an example embodiment of the invention.

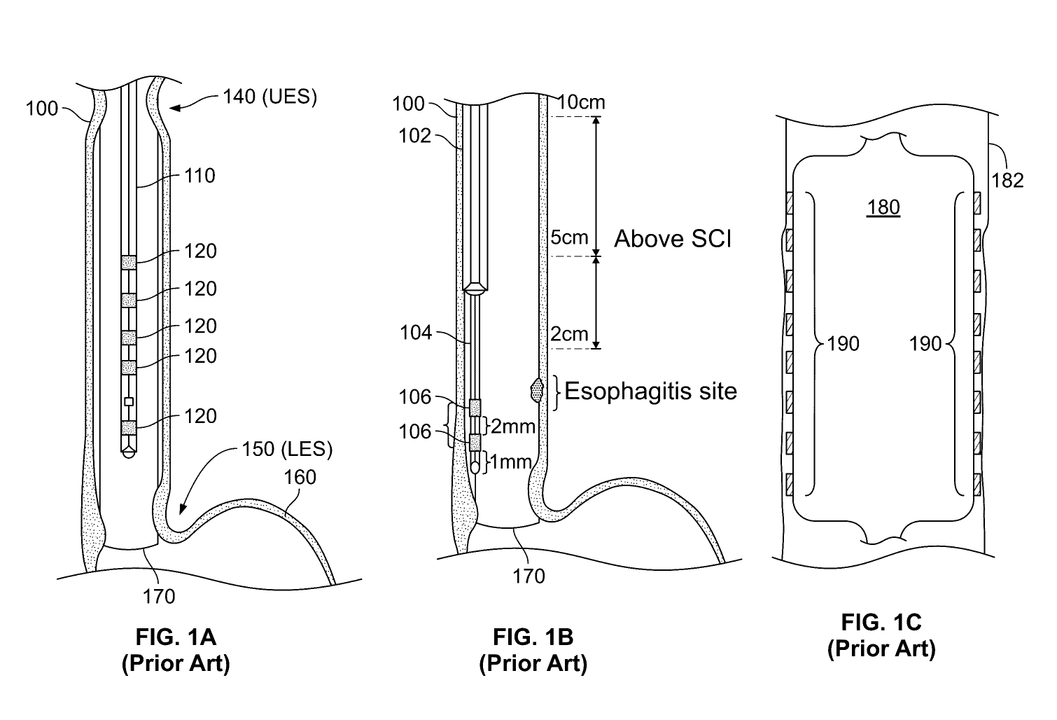

### DETAILED DESCRIPTION

The description that follows provides various details of exemplary embodiments. However, this description is not intended to limit the scope of the claims but instead to explain various principles of the invention and the manner of practicing it.

Unless explicitly stated, the embodiments of methods described herein are not constrained to a particular order or sequence of steps, operations or procedures. Additionally, some of the described method embodiments or elements thereof can occur or be performed at the same point in time.

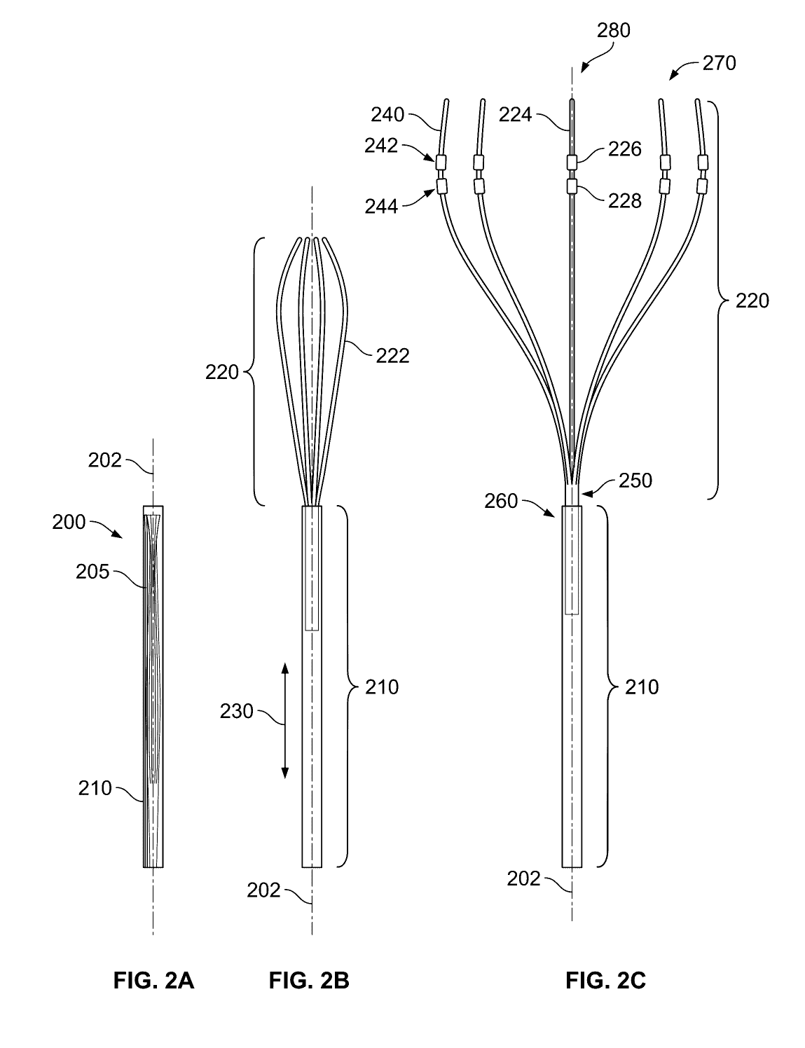

FIGS. 2A-2C show an MI device 200 in various deployment states (e.g., tines are fully housed in a retaining member, partly deployed, and fully deployed) according to an example embodiment of the invention. FIG. 2A shows the tines 222 completely stowed (retained or contained) in retaining member 210 (e.g., a sheath or other structure). In FIG. 2B most of the length of tines 222 is deployed (the remaining length thereof is still retained in retaining member 210), and in FIG. 2C, tines 222 are fully deployed.

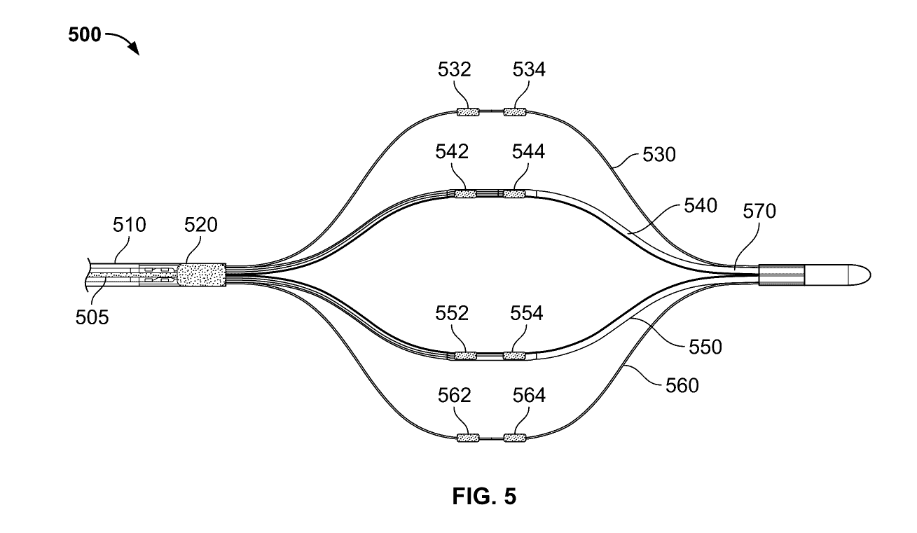

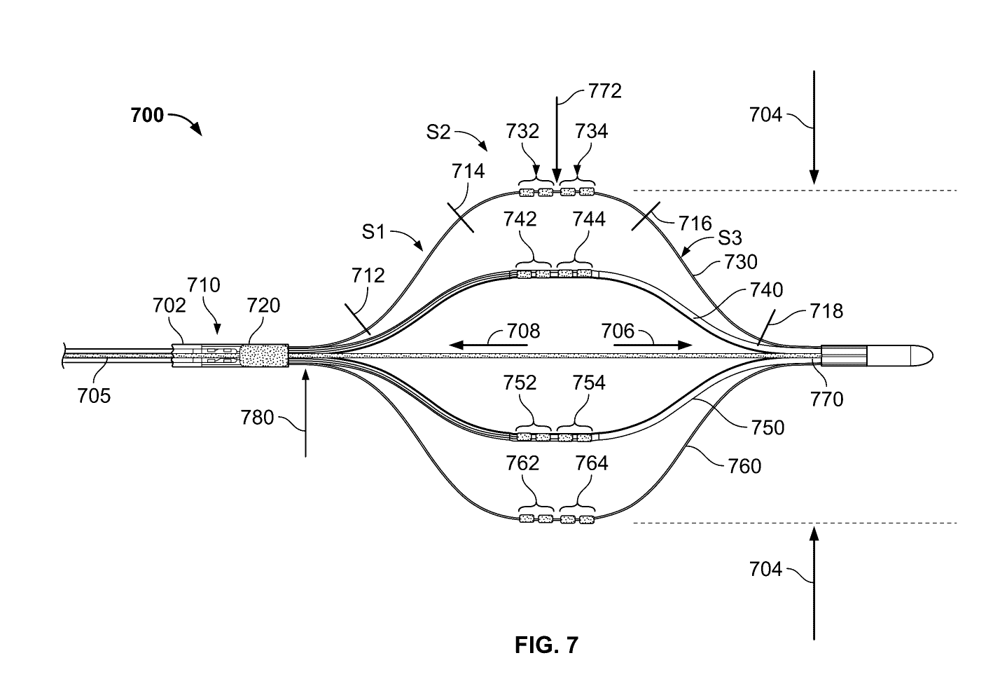

According to this embodiment the MI device may include an open-sided flexible tines structure 220 that may include N flexible tines, or ribs, or rib like strips such as tine 222. A same end of the N metal flexible tines or ribs are firmly tight together at trailing end 250 of the tines structure. Trailing end 250 of the tines structure is shown just outside, or adjacent, distal end 260 (‘neck’ 260) of retaining member 210, while the other end of the structure including the N flexible tines or ribs (e.g., leading end 270) is open (280) due to the flexible tines or ribs being pre-conditioned, or pre-shaped, to deflect laterally outwardly, to ‘open up’, when no external force is exerted on them. (For example, this force could be applied retaining member 210 when it fully houses the tines, or by the esophagus during impedance measurement.) In other embodiments both the trailing end and leading end of a tines structure may be closed, as illustrated, for example, in FIG. 5 and in FIG. 7, which are described below. Distal end 260 may be a collar that holds together tines 222 and may, therefore, part of the tine structure 220, although this structure could be built without such a collar.

MI device 200 may have an outer retaining member 210 (e.g., a sheath, a lumen) which is configured to be deployable and retractable through a working channel of an endoscope. Retaining member 210, by being deployable through the working channel of the endoscope, may ‘extend’ (make longer) the operational length of the working channel of the endoscope. The internal diameter (d) of the endoscope's working channel may, for example, be 2.8 mm and the largest diameter of the sheath will have a size less than this, allowing it to be used with many standard endoscopes. The endoscope working channel may have other internal diameters, for example less than 2.8 mm, or greater than 2.8 mm, and retaining member 210 may be designed accordingly.

During introduction of MI device 200 to the patient (e.g., during insertion of device 200 through, for example, the esophagus, or through trans-nasal placement), MI device 200 may be in a ‘stowed’ state in which retaining member 210 completely houses the tines/strips, as illustrated in FIG. 2A. After introduction of MI device 200 (e.g., the device is, at this stage, at or near the esophageal site whose impedance is to be measured), the structure of the N flexible tines, or rib like strips, may be slowly pushed out in and deployed; e.g., by using AAM 205, through retaining member 210, as illustrated by FIG. 2B, until the N metal flexible tines, are deployed to an operational state, as illustrated by FIG. 2C (By way of example, FIG. 2B and FIG. 2C respectively show four and five flexible tines). Alternatively, retaining member 210 may be inserted (230) to a location of interest in the esophagus with the tines fully housed by the retaining member, and then the retaining member may be retracted backward (230) to expose/deploy the tines. In some embodiments, flexible tines structure 220 may be introduced in additional ways, for example trans-nasally, trans-orally, etc., and, in doing so, flexible tines structure 220 may not necessarily require a (separate or dedicated) retaining device (e.g., sheath) in order to be introduced. (For example, the retaining function used to retain flexible tines structure 220 may be done by a working channel of an endoscope.)

On each tine or rib like strip (e.g., tine 224, FIG. 2C) may be mounted multiple impedance electrodes (e.g., electrodes 226 and 228 on tine 224), for example at the distal end of the tine. Impedance electrodes 226 and 228 of tine/strip 224 may make an electrode pair that may be used to measure the impedance (Z) of a tissue area at, near, or in-between these two electrodes. Each tine may include one or more pair of impedance electrodes. By way of example, tine/strip 224, FIG. 2C, includes one pair of impedance electrodes (electrodes 226,228), tine/strip 240, FIG. 2C, also includes one pair of impedance electrodes (electrodes 242,244), and so on. Each tine or rib like strip may include M (M>1) pairs of impedance electrodes that may axially sense mucosal impedance in multiple points or areas (e.g., a point/area per pair of electrodes) along the length of the esophagus. The greater N (the number of tines in a tines structure) and the greater M (the number of impedance electrode pairs on/in each tine), the greater the esophageal tissue area (both peripherally and axially/lengthwise) whose impedance can be measured. As shown in, for example, FIG. 4, and described below, electrodes making up a electrode pair may be located on separate tines.

An esophageal impedance may be measured using not only pairs of electrodes that are located on the same tine, but alternatively, or additionally, using electrodes on separate or different tines. Esophageal impedance(s) may be measured between (using) one or more electrodes in one tine and one or more (paired) electrodes in other one or more separate tines. An electrode on any tine may be used with other electrodes to measure lateral impedance(s) or lengthwise impedance(s), or diagonal impedance(s) or all three types of impedances; that is, lateral and diagonal and lengthwise impedances. That is, a particular electrode located in/on a first tine may operationally be paired (make a pair) with another electrode, adjacent or not, on the same tine to measure a lengthwise impedance along the length of the lumen (e.g., esophagus). The particular electrode may also be paired with another electrode on a different tine (adjacent to the tine the particular electrode is on, or not) to measure a lateral or diagonal impedance of the lumen (e.g., esophagus). A pair of impedance electrodes on a same tine may be adjacent (with no intervening electrode(s)), or spaced away, with one or more other electrodes interposed between them. A particular electrode may participate in (be factored into) multiple impedance measurements, where each measurement involves pairing the same particular electrode with another electrode on the same tine or on any other tine, to thereby measure one or more lengthwise impedances and/or one or more lateral impedances and/or one or more diagonal impedances. A switching circuit may and/or may be configured to be electrically connected to all the electrodes on all the tines, and include a controller to switch between electrodes to select the electrodes for measuring the required, desired, or intended impedances, be them lengthwise impedances, lateral impedances or diagonal impedances.

Retaining member 210 may also act to mechanically deform (e.g., collapse or flatten) the tines such that they snuggly fit inside retaining member 210, and hence, for example, inside the working channel of an endoscope. The tines may be of a suitably elastic material that may be chosen specifically for its mechanical properties, specifically the elastic modulus and yield strength. The tines may be pre-conditioned with a shape which provides a spring tension force in the radial outward direction (e.g., towards the esophageal mucosa tissue). The tines or ribs may be retained by and in retaining member 210 until after intubation, when the process of impedance measurement is, or may be, commenced. The preconditioned shape and elasticity of the tines may be chosen such that when retaining member 210 is retracted (to expose the tines and impedance electrodes), or (depending on the configuration) when the tines are pushed through and out of the retaining member, the tines ‘carrying’ the impedance electrodes may expand radially outward (with respect to a longitudinal axis 202 of the retaining member or MI device as a whole) such that the impedance electrodes apply an operationally suitable contact force, or pressure, on the inner wall of the esophagus for a range of diameters of the esophagus. Each pair of impedance electrodes may measure impedance at a point or area of the esophageal mucosa. Retaining member 210 may be a working channel of an endoscope.

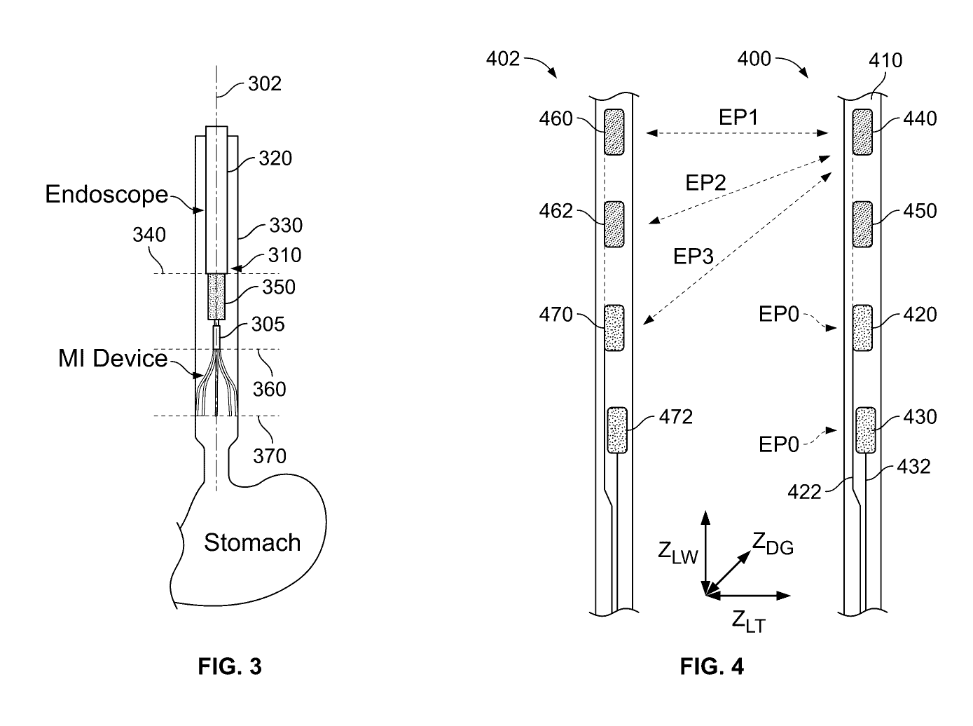

FIG. 3 shows a MI device similar to the MI device of FIGS. 2A-2C, which is deployed in the esophagus according to an example embodiment of the invention. During operation, distal tip 310 of endoscope 320 may be inserted through esophagus 330 until distal tip 310 reaches location 340. Location 340 represents an approximate location (in the esophagus) suitable for deploying the MI device and positioning it at an appropriate location relative to, for example, a reference location (or relative to a reference object) on or in, the esophagus. The reference location may be, for example, the distal esophagus (e.g. a specified distance from the lower esophageal sphincter (“LES”) or relative to the Squamocolumnar (SC) junction), or relative to any other suitable or convenient reference location. (The Squamocolumnar (SC) junction, or “Z-line”, represents the normal esophago-gastric junction where the squamous mucosa of the esophagus and columnar mucosa of the stomach meet.) At this point, retaining member 350, with the tines residing in it, may be pushed (e.g., by using an AAM) through a working channel of or in endoscope 320 until the distal end of the retaining member reaches line 360. Then (after the distal end of retaining member 350 is in place), the tines may be pushed through and out of retaining member 350, for example until they reach line 370, in order to have the impedance electrode pairs deployed (“naturally” deflected radially/laterally outwardly, or open-up) in the desired position. In some embodiments, retaining member 350 may be an integral part, or an extension, of an endoscope (e.g., part of a working channel of the endoscope). (At 305 is shown a collar that holds the tines together.)

In some embodiments, the retaining member may be pushed, with the tines stowed or residing in it, through the endoscope's working channel until the distal end of the retaining member reaches a desired location in the esophagus. Then, the retaining member may be retracted backward in the endoscope's working channel (while keeping the axial position of the tines approximately constant via an AAM similar to AAM 205) to deploy the tines such that they are radially deployed (“naturally” deflected radially/laterally outwardly, or open-up) in the desired position with respect to a longitudinal axis 302 of the retaining member or MI device as a whole, such that they push the impedance electrodes against an inner wall of the esophageal mucosa with an operational force. The term ‘operational force/pressure/contact’ refers herein to a force that is high enough to obtain useful impedance measurements, but low enough in order not to inflict damage to the measured/monitored esophageal tissue.

Once operational contact between the impedance electrodes and the esophageal mucosa is achieved, the MI device may register a drop in impedance (Z) values, from the effectively infinite resistance characterizing the “open” circuit which is the state that is typically registered by the impedance electrodes prior to being in contact with the esophageal mucosa. During the time when operational contact between the impedance electrodes and the esophageal mucosa is maintained, the measured impedance may initially drop to relatively low impedance values that typically characterize the esophageal mucosa, and stabilize. At this point, accurate impedance data, which represents or is related to mucosal impedance at multiple locations on the inner circumference and along the length of the esophagus, may be collected.

By way of example, the tines of the MI device may exert a minimum operational force of 0.1N, at the impedance electrodes, on the internal surface of the esophageal mucosa when the MI device's retaining member is retracted to operationally deploy the tines. Stainless Steel 400 may be used as the tine material, as it is a readily available material with elongation and yield properties appropriate for the intended design. Taking the known minimum force requirements at a known range of diameters together with specified material properties, computer aided design software may be used to approximate mechanical properties (e.g., curvature, width and thickness) of the tines that can establish appropriate contact with the walls of the mucosa, while still allowing for the tines to be compressed into the retractable retaining member without permanently deforming (yielding) the tines.

As described herein, the MI device deployment method is to position impedance electrode(s) at a known (approximately) position, for example, relative to (above) the LES or SC junction. The LES may not be visible endoscopically but may be identified by other means (e.g. by using esophageal manometry where position of the LES is identified relative to Nares). The SC junction (a more or less a circumferential line in the esophageal mucosa) is typically visible endoscopically and may be used to identify proper location.

In the open-ended tines structure (e.g., FIGS. 2A-2C, FIG. 3), various MI device positioning methods may be used, including lining the tip of the tines up with the SC line and then withdrawing the endoscope, and/or device relative to the endoscope, a specified distance. Ruled markings may be provided on the retaining member or AAM of or related to the MI device, or a direct marker near the closed tip of the device, to line up with or be offset some desired amount from the SC junction at the time when measurements are taken.

FIG. 4 shows part of an example flexible tine 400 according to an embodiment of the present invention. A tine of a MI device (e.g., tine 400) may be, or include, an elongated body (e.g., elongated body 410). The elongated body may be made of metal (e.g., Stainless Steel) or electrically non-conductive material (e.g., plastic). By way of example, elongated body 410 may include one pair of impedance electrodes (e.g., e.g., electrode pair EP0 formed by example electrodes 420,430), or more than one electrode pair; for example it may include two separate pairs of impedance electrodes (e.g., electrode pair {420,430} and electrode pair {440,450}), three separate pairs of impedance electrodes, and so on.

An electrode located on tine 400 may operationally form (be paired with) an electrode pair with (that is, it may be operationally paired or used in conjunction with) any other electrode, and/or it may be paired with multiple electrodes (to form multiple electrode pairs) on the same tine. For example, electrode 420 may be operationally paired only with electrode 430, or only with electrode 440, or only with electrode 450, etc. In another example, a particular electrode on a particular tine may be operationally paired with some other electrodes on the same particular tine, then (or simultaneously) with another electrode on the same particular tine, then (or simultaneously) with yet another, different, electrode on the same particular tine, etc. For example, an electrode on tine 400, for example electrode 420, may be operationally paired, during a same impedance measurement procedure, first with electrode 440, then (or simultaneously) with electrode 450, and then (or simultaneously) with electrode 430 to measure, in this example, three lengthwise impedances along the length of tine 400.

Tine 402 may be structurally similar to tine 400, and it may include the same number of electrode pairs as tine 400 (e.g., two electrode pairs: a first electrode pair {460,462} and a second electrode pair {470,472}. Electrodes 460, 462, 470 and 472 of tine 402 may be paired in a similar manner as the electrodes of tine 400, or differently, to also measure lengthwise impedances, this time along the length of tine 402. In another example, ‘cross-tines’ electrode pairs may be formed; that is, an electrode in tine 400 or in tine 402, may be paired with an electrode, or with electrodes (for example with two electrodes or three electrodes), located on another tine, so that electrode pairs may be formed between tines. For example, electrode 440 on tine 400 may be paired with one electrode on tine 402 (e.g., with electrode 460, to form an electrode pair ‘EP1’), or with multiple electrodes on tine 402 (e.g., with electrodes 462 and 470; to respectively form electrode pairs ‘EP2’ and ‘EP3’). Any electrode on any tine may be paired with any other electrode, or electrodes, on any other tine or tines of the tines structure. Multiple electrodes on a particular tine may be paired with the same electrode(s) on another tine or other tines, or with different electrodes located on the same tine or on different tines, etc. This way, multiple lengthwise impedances (FIG. 4, Z_{LW}), lateral impedances (FIG. 4, Z_{LT}) and diagonal impedances (FIG. 4, Z_{DG}) may be measured to obtain an multidirectional impedance map (along the surface of the esophagus) representing the esophageal mucosal impedance in the region of study.

The impedance electrodes (e.g., impedance electrodes 420 and 430) may be, for example, mounted in or on a flexible tine elongated body 410. If elongated body 410 is made of electrically conductive material, the impedance electrodes may ‘sit’ (mounted) in an insulating layer or in an insulated socket or ‘pocket’ that may be attached or affixed to the tine elongated body, such that the impedance electrodes and the tine elongated body are electrically insulated. Each impedance electrode may be connected to, for example, a remote monitoring system (not shown in FIG. 4) via an electrical conductor. For example, impedance electrodes 420 and 430 may respectively be connected to a remote system via electrical conductors 422 and 432. A non-conducting layer (e.g. polymer coating) may be used to electrically isolate the conductive surface of the electrodes' circuit, so that only the electrodes themselves (and not the circuit's conducting traces) are electrically exposed to contact with the esophageal surface (mucosa).

FIG. 5 shows an MI device 500 according to another example embodiment of the invention. MI device 500 may include a retaining member 510 (e.g., a sheath) with distal end 520. (Distal end 520 may be or include a collar that holds the tines together.) A tines structure, which is part of MI device 500, may include N flexible tines or ribs. The N flexible tines or ribs may initially be stowed in (e.g., housed by) retaining member 510 such as when the MI device is in a ‘standby’ state or during intubation, though in FIG. 5 the flexible tines or ribs (four example tines, designated as 530, 540, 550 and 560, are shown in FIG. 5) are shown deployed. Retaining member 510 may be a working channel of an endoscope.

Tines 530, 540, 550 and 560 may be deployed at a desired location in the esophagus either by inserting the entire MI device (e.g., the retaining member with the tines fully compressed\stowed in it) to the intended location and, then, retracting the retaining member backwards to expose the tines, or inserting the retaining member, with the tines fully compressed by the retaining member, to an intended location and, then, pushing the tines through and out of the retaining member. A AAM 505 may be used to actuate (‘push-pull’) the tines relative to retaining member 510. For example when used in an endoscope working channel, both retaining member 510 (if used) and AAM 505 will extend out to the proximal (controlling end) of the endo scope so that they may be manipulated separately or together depending on what action or procedure phase is desired.

Tines 530, 540, 550 and 560 may respectively include (e.g., have mounted therein or thereon) impedance electrode pairs such as {532,534}, {542,544}, {552,554} and {562,564}. All the tines, or some of them, may have more than one pair of impedance electrodes. Different tines may have different numbers of pairs of impedance electrodes. Tip 570 may include a marker to assist in axial position of the device in the esophagus, e.g. at a desired axial distance relative to a predetermined reference point, line or area. For example, the Squamocolumnar (SC) Junction (esophago-gastric junction), or LES may be used as a reference point.

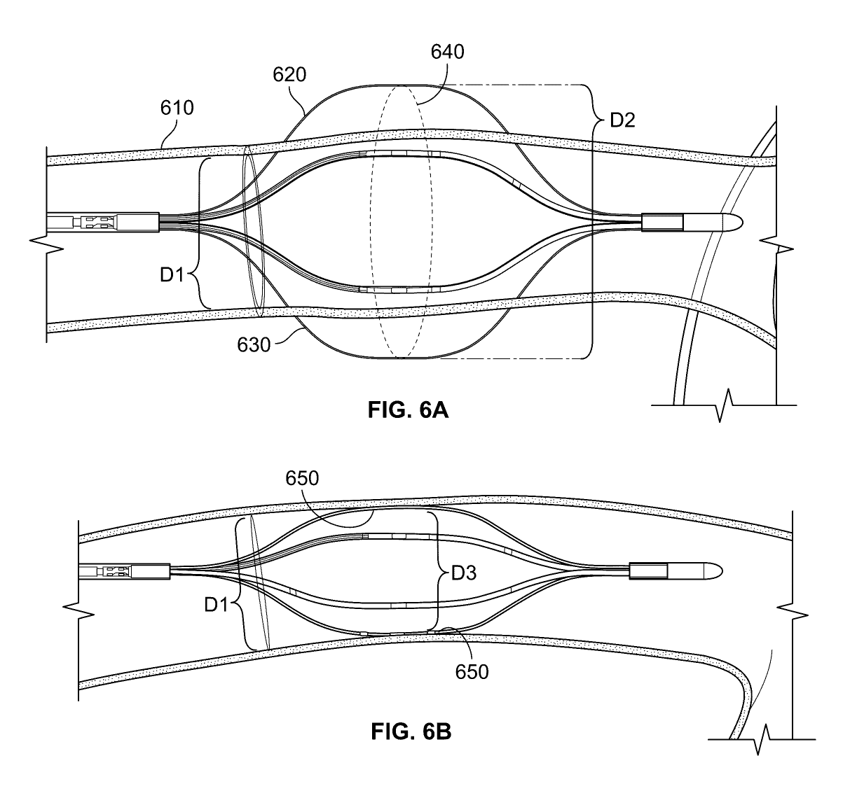

FIG. 6A comparatively shows a typical relationship between a ‘closed-sided’ (a ‘cage’ like) MI device similar to MI device 500 in a “natural”/released, stress-free, state relative to a size of an esophagus 610. The spring force exerted by the preconditioned shape of the tines is directly proportional to the amount of deflection, deviation or displacement from the “natural” (stress free) diameter of the device. Therefore, the device in its free state is designed to expand larger than the inner diameter of the esophagus, such that an operational contact can be made with the esophageal mucosa.

For illustrative purposes, a section of esophagus 610, full with ‘content’ (e.g., bolus, the MI device itself, etc.), may have a representative diameter or width D1, which may typically be within the range of 18 mm-34 mm. A diameter D2 of a circle (640) (which may be measured, for example, between tines 620 and 630) circumscribing the tines of the closed-sided MI device in a stress-free (uncompressed or ‘free’) state is preferably greater than diameter D1 of the esophagus. The diameter difference (D2−D1), in conjunction with the mechanical properties of the tines, may be calculated such that, when the MI device is in operation, the tines, having a compressed operational diameter D3 (FIG. 6B, D3<D2), apply at least a minimal force (spring force) on esophageal mucosa 650 that is required for reliable/useful measurement of the esophageal tissue impedance.

The tines may be made of metal, or plastic or other non-metallic materials or any combination thereof and designed to have a free, “rest”/“released” or “natural” (force-free) shape and flexibility, that will impart appropriate contact forces at the impedance electrode locations suitable for measuring the impedance of the esophageal mucosa when deflected inward to the range of representative esophageal radii. Acting as a spring, the ‘spring’ force that a tine applies to the esophageal tissue is generally proportional to the tine's/rib's radial inward displacement. In general, the larger the esophagus's diameter, the lesser the displacement of a tine, and the lesser the spring force applied by the tine. For example, using a prototype MI device it was found (by interpolation of data) that with an esophagus diameter of approximately 30 mm, the force that the tines applied was 0.29N, and with an esophagus diameter of approximately 20 mm, the force that the tines applied was 0.55N.

It is desired that the contact force variability through the range of esophageal diameters be limited to assure that appropriate contact forces, between electrodes and the tissue, are sufficient to provide for reliable impedance measurements while not being excessive such that excessive distension of the esophagus, patient discomfort, or even tissue damage may result. Given the data described above, the ratio between the two contact force extremes is relatively small at 1.9 (0.55N/0.29N≅1.9). To obtain the required contact force range, the free diameter of the tines structure is designed along with the mechanical properties (e.g., spring constant) of the tines in such a way that contact force range is minimized through the operational range of esophageal diameters. In addition, the shape and material properties of the tines are designed such that they do not permanently deform (yield) when retracted inside the retaining member, which could adversely altering their operational contact force characteristics.

The MI device of the present invention may be adapted for use in conjunction with an endoscopic procedure to quickly and easily obtain impedance measurements from multiple circumferential and axial locations in the esophagus. Regardless of method of introduction into the esophagus, measurements may be processed for indications of mucosal damage that are indicative of damage due to, for example, gastrointestinal reflux disease (GERD), non-erosive reflux disease (NERD), Barrett's esophagus, and injuries.

FIG. 7 shows an MI device 700 according to another example embodiment of the invention. MI device 700 may include a retaining member 710 with distal end 720. A tine structure of MI device 700 may include N flexible tines or ribs. The flexible tines or ribs may initially be stowed in retaining member 710 (e.g., a sheath) such as when the MI device, or the tines structure thereof, is in a ‘stowed’ state or during intubation, though in FIG. 7 the flexible tines (for example tines, designated as 730, 740, 750 and 760, are shown in FIG. 7) are shown deployed. Retaining member 710 may be a working channel of an endoscope. (Distal end 720 may be or include a collar for holding the tines together.)

Tines 730, 740, 750 and 760 may be deployed at a location of interest in the esophagus either by inserting the entire MI device (e.g., the retaining member with the tines stowed in it) to the intended location and, then, retracting the retaining member backwards, for example in the endoscope's working channel, to disclose the tines, or inserting the retaining member, with the tines stowed in the retaining member, to an intended location and, then, pushing the tines through and out of the retaining member. Positioning of the electrodes in the esophagus may be adjusted by some combination of the above-described actions and by sliding the deployed or partially deployed MI device axially or distally within the esophagus.

Each of the tines 730, 740, 750 and 760 may respectively include (have mounted therein or thereon), for example, two impedance electrode pairs {732,734}, {742,744}, {752,754} and {762,764}. All the tines, or some of them, may have one pair of impedance electrodes, or more than one pair of impedance electrodes. Different tines may have different numbers of pairs of impedance electrodes. As described herein, for example in connection with FIG. 4, an electrode on a particular tine may be paired with an electrode that is located on the same particular tine and/or with an electrode that is located on another tine. The number of electrodes located on a particular tine does not necessarily have to be the same as the number of electrodes in another tine.

MI device 700 may include an axial actuating member (AAM) 702 to move the tines structure relative to retaining member 710. MI device 700 may also include a force adjusting member (FAM) 705 to adjust the contact force, or pressure, that the impedance electrodes apply on the esophageal tissue, by adjusting the operational diameter 704 of the tines structure by pushing or pulling leading end 770 of the tines structure by AAM 702. AAM 702 may be contained, at least partly, in retaining member 710 and be movable, for example by a physician, in direction 706 or in direction 708 to adjust the contact force that the spring tines 730-760 apply on the esophageal lumen (or other lumen). AAM 702 may also enable, for example, a physician to distally push leading or distal end 770 in direction 706 in order to flatten out the tines structure to facilitate stowing it in the retainage member (e.g., a sheath, a working channel of an endoscope, and the like.) A tines structure may be stowed in, and deployed from, a retaining member, which may be a relatively short annular member, rather than using a (e.g., ‘full length’) sheath per se, thus enabling rendering a sheath or endoscopic working channel unnecessary. When used in the working channel of an endoscope (or an axially oriented annular open channel of any suitable tubular instrument), the working channel of the endoscope, or tubular instrument, may be used as the retaining member and so the MI device itself does not necessarily include or require a retaining member. A removable short length retaining member (e.g., a short tubular instrument) may be provided to (e.g., temporarily) retain the tines (keep them in a stowed state) until the MI device is inserted into an endoscope's working channel.

The impedance electrodes mounted on the tines may be electrically connected to an external impedance measuring system via electrical wires that may pass through retaining member 710, for example through AAM 702.

Tines may have mechanical characteristics (e.g. flexibility in bending) that change along their length. A tine may have S segments (S=1, 2, 3, . . . ,), each segment Si with varying geometry and/or material property characteristics to yield different bending stiffness (Ki), to thereby control the operational contact force of the electrodes with the esophageal mucosa and to make such force relatively uniform among the various electrodes for the design range of esophageal diameters. The varying geometry and/or mechanical characteristics and/or material property among the segments may also be designed to minimize or eliminate yielding of the tines when stowed in the retaining member.

The geometry and/or material and/or mechanical properties of a tine may be set to such values that an electrode(s) segment, Si, including electrodes is made less flexible (e.g. it has greater or higher bending stiffness) than an adjacent segment, meaning that the force required to deflect an electrode(s) segment a given distance is greater than the force required to deflect the segment adjacent to the electrode(s) segment. For example, the geometry and/or material and/or mechanical properties characterizing segments S1, S2 and S3 (FIG. 7) may be set such that the bending stiffness of segments S1 and S3 (K1, K3, respectively) is/are lower than the bending stiffness of segment S2 (K2). That is, by selecting suitable geometry and/or material and/or mechanical properties for tine segments S1, S2 and S3 (for example), segments S1 and S3 can be made more prone to bending under pressure than segment S2. A tine may have a geometry and/or material and/or mechanical properties that changes gradually, alternately or linearly along its length. The bending stiffness of such a tine may, for example, increase gradually or linearly from the tine's proximal end 780 towards the tine's point (e.g., point 772 of tine 730), and decrease from that point towards the tine's distal end 770. Having tines with varying flexibility is beneficial in terms of, for example, manipulation of the contact force that the tines apply on the esophageal tissue.

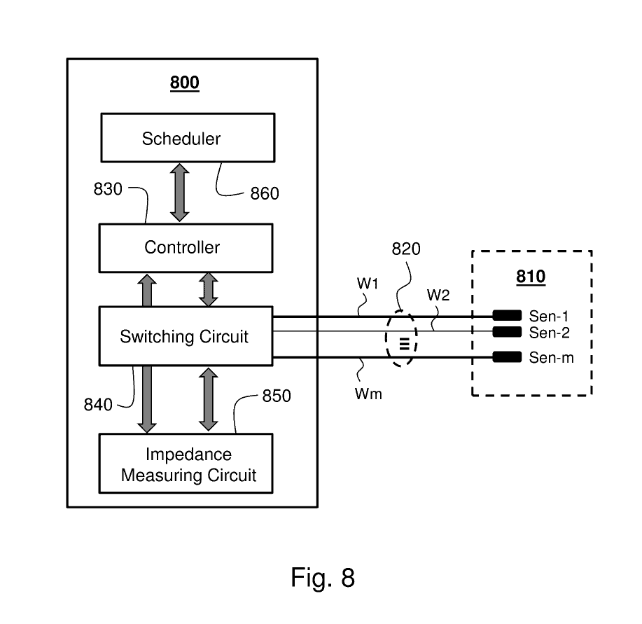

FIG. 8 shows a block diagram of an impedance measurement control system 800 according to an embodiment of the invention. Control circuit 800 may be connected to a mucosal impedance device 810 via an electrical cable 820. (Only the electrodes of mucosal impedance device 810 are shown in FIG. 8.) Control circuit 800 may include, among other things, a controller 830, a switching circuit 840 and an impedance measuring circuit 850. Controller 830 may operate switching circuit 840 to select electrodes, from mucosal impedance device 810, for impedance measurements, and control impedance measuring circuit 850 to measure impedances between pairs of electrodes that are selected by controller 830. Every electrode on every tine of a tines structure associated with mucosal impedance device 810 may be electrically connected to control system 800 via an electric wire (e.g., wires 422 and 432, FIG. 4). In FIG. 8, a number of m impedance electrodes, designated as Sen-1, Sen-2, . . . , Sen-n, of mucosal impedance device 810 are respectively connected to switching circuit 840 via electric wires W1, W2, . . . , Wm.

Impedance measuring circuit 850 is configured to measure impedance between two on-board electric terminals. Depending on a particular pair or set of electrodes that controller 830 selects for impedance measurement, controller 830 controls switching circuit 840 to select and connect the pertinent wires to the electric terminals to measure impedance between the selected electrodes. Controller 830 may optionally generate an impedance map of, for or representative of a monitored mucosa (e.g., esophageal mucosa) from impedance data representing or derived from the impedance measurements. The wires electrically connecting all the electrodes in the tines structure to the control system (e.g., to the switching circuit) may pass through, on, or around the retaining member (e.g., retaining members 210, 350, 510 and 710), or any other part of the device. The circuit shown in FIG. 8 may include additional circuitry, for example supporting data collection circuits/devices, for example, microprocessors, displays, batteries, memory, etc. Impedance measurement control system 800 may include a scheduler 860, synchronizer or time table to indicate to controller 830, or to be used by the controller, to determine which electrodes on which tines should be used, and when.

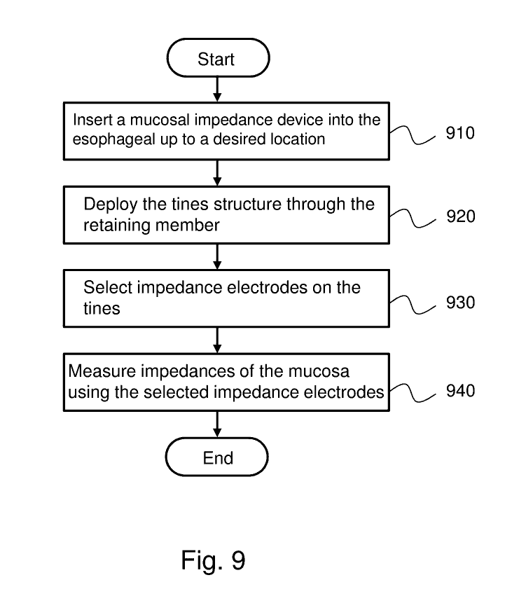

FIG. 9 shows a method for measuring impedance of an esophageal mucosa by using a mucosal impedance device (e.g., device 810, FIG. 8) that is connected to an impedance measuring system (e.g., system 800, FIG. 8), and that includes a retaining member and a tines structure initially stowed in the retaining member, where the tines structure includes a number N of flexible tines, where each tine has mounted thereon impedance electrodes. At step 910 insert the mucosal impedance device into the esophageal up to a desired location. At step 920 deploy the tines structure through the retaining member to expand the tines structure radially outward with respect to a longitudinal axis of the retaining member, to thereby make contact between the impedance electrodes and an esophageal mucosa. At step 930, select impedance electrodes (for example by controller 830 of FIG. 8) on the tines, for measuring impedance, and at step 940, measure (for example by controller 830 of FIG. 8) impedances of the mucosa using the selected impedance electrodes. Steps 930 and 940 may be reiterated or repeated or iterated for additional impedance measurements.

Controller 830 may be for example a central processing unit (CPU) executing software, or may be dedicated circuitry, and thus may be configured to carry out methods as described herein by for example executing code or instructions, or acting according to dedicated circuitry. Similarly, other modules or circuits, such as scheduler 860, switching circuit 840 and impedance measuring circuit 850, may be implemented by or in one or more CPUs and/or using dedicated circuitry.

Step 910 may include moving the impedance measuring device in the retaining member by an axial actuating member (AAM) to adjust the location of the impedance measuring device. Step 920 may include adjusting a contact force of the tines by using a force adjusting member (FAM).

Multiple impedance measurements may be taken (e.g., to produce an impedance map by the controller), for example by system 800 (e.g., by controller 830, FIG. 8), some of which may be lengthwise measurements resulting in lengthwise impedances, others may be lateral measurements resulting in lateral impedances, and others may be diagonal measurements resulting in diagonal impedances. To implement this ‘crisscross’ impedance measurement scheme, controller 830 (for example) may use a scheduler (e.g., scheduler 860), synchronizer or time table to determine which electrodes on which tines should be used, and when. The controller may reiterate steps 930 and 940 for additional impedance measurements, for example based on information that stored in the scheduler, synchronizer or time table.

The articles “a”/“an” are used herein to refer to at least one) of the grammatical object of the article, depending on the context. For example, “an element” can mean one element or more than one element. The term “including” is used herein to mean, and is used interchangeably with, the phrase “including but not limited to”. The terms “or” and “and” are used herein to mean, and are used interchangeably with, the term “and/or,” unless context clearly indicates otherwise. Having described exemplary embodiments of the invention, it will be apparent to those skilled in the art that modifications of the disclosed embodiments will be within the scope of the invention. The present disclosure is relevant to various types of catheters that use a balloon to measure pressure or other parameter. Hence the scope of the claims that follow is not limited by the disclosure herein.

1. A device for measuring an impedance of an esophageal mucosa, comprising:
a tines structure comprising a number N of flexible tines, said tines structure retainable in and deployable through a retaining member, each flexible tine having mounted thereon one or more impedance electrodes,
wherein the N tines are preconditioned with a shape and mechanical properties such that when the tines structure is deployed, the preconditioned shape and mechanical properties cause the N tines to expand radially outward with respect to a longitudinal axis of the retaining member, to measure impedances of an esophageal mucosa.

2. The device as in claim 1, further comprising the retaining member.

3. The device as in claim 1, wherein the retaining member is selected from the group consisting of an external tube, a sheath, a tubular instrument, and a working channel of an endoscope.

4. The device as in claim 1, wherein the tines structure is selected from the group consisting of: an open-sided tines structure and a closed-ended tines structure.

5. The device as in claim 1, wherein the closed-ended tines structure further comprises an axial actuating member (AAM) for moving the tines structure relative to the retaining member.

6. The device as in claim 1, wherein the closed-ended tines structure further comprises a force adjusting member (FAM) for adjusting a force that the tines apply to the esophageal mucosa.

7. The device as in claim 1, wherein the tines are made of or include metal or plastic.

8. The device as in claim 1, wherein an impedance electrode on a particular tine is operationally paired with: (1) an impedance electrode on the same particular tine, to measure a lengthwise impedances along the length of the tine, and/or (2) an impedance electrode on another tine to measure a lateral impedance between the tines, and/or (3) an impedance electrode on another tine to measure a diagonal impedance between the tines.

9. The device as in claim 1, wherein a bending stiffness (K) of a tine changes gradually, alternately or linearly along the tine.

10. The device as in claim 1, wherein a tine of the N tines comprises S tine segments, at least some of the S tine segments having different bending stiffness (K).

11. The device as in claim 10, wherein a bending stiffness Ki of a particular tine segment Si comprising an impedance electrode is greater than a bending stiffness of a tine segment adjacent to the particular tine segment Si.

12. A control system for measuring mucosal impedances, comprising:
a switching circuit configured to be electrically connected to electrodes mounted on a mucosal impedance device;
an impedance measuring circuit for measuring impedances between impedance electrodes, the impedance electrodes selectively connected to the impedance measuring circuit via the switching circuit; and
a controller configured to: (i) control the switching circuit to select impedance electrodes, and (ii) control the impedance measuring circuit to measure impedances between the selected electrodes.

13. The control system as in claim 12, further comprising a scheduler to indicate to the controller the impedance electrodes that the controller should select.

14. A method for measuring an impedance of an esophageal mucosa, comprising:
for a mucosal impedance measuring device utilizing a retaining member and a tines structure stowed in the retaining member, the tines structure comprising a number N of flexible tines, each tine having mounted thereon impedance electrodes,
(i) inserting the impedance measuring device into the esophageal, up to a desired location;
(ii) deploying the tines structure through the retaining member to expand the tines structure radially outward with respect to a longitudinal axis of the retaining member, to thereby make contact between the impedance electrodes and an esophageal mucosa; and
(iii) selecting impedance electrodes; and
(iv) measuring impedances of the mucosa using the selected impedance electrodes.

15. The method as in claim 14, further comprising reiterating steps (iii) and (iv) for additional impedance measurements.

16. The method as in claim 14, where step (i) comprises moving the impedance measuring device in the retaining member by an axial actuating member (AAM) to adjust the location of the impedance measuring device.

17. The method as in claim 14, where step (ii) comprises adjusting a contact force of the tines by using a force adjusting member (FAM).

18. The method as in claim 14, wherein selecting the impedance electrodes on the tines comprises any of: (i) selecting impedance electrodes for measuring lengthwise impedance measurements, (ii) selecting impedance electrodes for measuring lateral impedance measurements, and (iii) selecting impedance electrodes for measuring diagonal impedance measurements.
